(19) United States
(12) Patent Application Publication
(10) Pub. No.: US 2005/0287680 A1
(43) Pub. Date: Dec. 29, 2005

(54) Multianalyte assay method

(76) Inventors: Srivatsa Venkatasubbarao, Torrance, CA (US); Ashutosh Sharma, Torrance, CA (US)

Correspondence Address: LAWRENCE S. COHEN;ATTORNEY AT LAW, SUITE 1220, 10960 WILSHIRE BOULEVARD, LOS ANGELES, CA, 90024, US

(21) Appl. No.: 10/876,405

(22) Filed: Jun. 25, 2004

(51) Int. Cl. C12Q 1/68; G01N 33/543; G01N 33/551

(52) U.S. Cl. 436/523

(57) ABSTRACT

A plurality of groups of colorimetrically distinguishable metal nanoparticles are prepared to label specific analytes whose presence in a sample is under investigation, each group for specific analytes. After being mixed with the sample so that labeling can occur if the analyte or analytes are present, the sample is exposed to a sensor having probes for the analytes under investigation. Binding of any of the analytes present will carry the metal nanoparticle as well, which then enables colorimetric detection of each label to determine which if any of the analytes is present in the sample. In an alternative method the probes can be labeled with calorimetrically distinguishable metal nanoparticle labels and any binding events can be detected calorimetrically.

### FIELD OF THE INVENTION

This invention relates to analyte assay apparatus and methods

### BACKGROUND

Nanotechnology is the science of creating functional materials and devices through nanometer scale control and exploitation of material's properties. Nanomaterials exhibit size dependent properties. One such nanomaterial of particular interest is colloidal gold nanoparticles. Gold nanoparticle have been widely used as biological labels in diagnostic test kits as well as in microscopy. Conventional in vitro tests such as lateral flow membrane strips use colloidal gold nanoparticles (CGNs) as colorimetric labels.

CGNs exhibit size dependent optical properties. However, individual nanoparticles are too small to be visible to the naked eye, and cannot be directly visualized. Therefore, the CGNs are precipitated to make them visible as thin films. Such CGN films exhibit optical properties of bulk gold and lose their size-dependant properties and behave as bulk gold.

CGNs consist of particles of gold from about 1 nm or smaller to about 250 nm, and exhibit size-dependent optical properties such as absorption at specific wavelengths, scattering and polarization. They appear orange, red, purple, or blue as the size of the particles change. The orange color of 3 nm or smaller particles is due to quantum size effects resulting from changes in electronic free path, due to the breakdown of conduction and valence bands into discrete levels. The size dependent color change of larger particles (>3 nm) is due to geometric effects and can be explained by the Mie theory of scattering.

Recent research shows that when CGNs are separated by a spacing of more than twice the particle radius (and a metal volume fraction (φ) of <10%), they will retain their individual properties In order to maintain CGNs at predefined spacing, CGNs are coated with a chemical that causes reduction of the dipole interactions between particles. One such chemical is silica used to create a shell of known thickness (Si-CGN). Silica reduces the dipole coupling between the individual particles, and thus the properties including the colorimetric property of the nanoparticles can be preserved.

The prior art does not include a system or method for detecting more than one analyte simultaneously using size-dependant colorimetric properties of nanoparticles.

### SUMMARY OF THE INVENTION

A method of multianalyte assay using shelled metal nanoparticles, in particular silica-shelled CGNs, in a plurality of selected discrete size groups having distinguishable colorimetric properties. The shelled metal nanoparticles in each size group are enabled for binding to specific analyte or analytes whose presence is under investigation in a sample and then labeled to the analytes, if present. Then the sample is assayed to a bioarray, the analytes, if present, binding to probes along with the size-dependant calorimetrically distinguishable shelled metal nanoparticles. At least two analytes are being assayed for and a calorimetrically distinguishable size group of shelled metal nanoparticles is labeled to each of the analytes.

The shelled metal nanoparticle, in particular CGNs can be made calorimetrically distinguishable by using the same size nanoparticles and different sized shells, or different sized nanoparticles, or both.

The silica-shelled CGNs in a group are preferably separated by a spacing of more than twice the CGN radius.

In an alternative method, the sample is first assayed with analytes, if present, biding to probes, and the plurality of size arrays of enabled metal nanoparticles, preferably enabled silica-shelled CGNs are exposed to the assay and will bind to the analyte for which each size group is enabled.

### DETAILED DESCRIPTION

The present invention exploits size-dependent colorimetric properties of metallic nanoparticles for multianalyte testing. The invention resides in a method for colorimetric assay of a plurality of analytes by use of size-dependant nanoparticle labels. Each of the plurality of specified size groups of metal nanoparticles is enabled to attach to a specific analyte or analytes. Then the sample is exposed to an assay bioarray for those analytes whose presence is under investigation. The binding of the analytes, if present, to respective probes will be observable due to the distinguishable colorimetric properties of the metal nanoparticle labels on each analyte since the metal nanoparticle size groups for each analyte are colorimetrically distinguishable. The process is useful in all types of assay in which binding of an analyte, if present, takes place upon exposure of the sample to a bioarray specified for the analytes under investigation. These include, antibody-antigen, DNA-DNA, protein-receptor, enzyme-inhibitor and other biomolecular and molecular binding events. In particular the invention resides in a method of tailoring or preserving the size dependent colorimetric properties of nanoparticles prepared due to molecular binding by spacing the nanoparticles apart in a molecular level.

The invention provides multianalyte detection. In one aspect it provides instrument free detection capability. When used with a reader it also provides the ability to quantify the analyte concentration. It provides the ability to measure its effects in reflection and/or transmission mode.

The invention in one aspect employs three basic elements.

One element is a sensor also referred to as a biochip, a bioarray, microarray, microchip, nanochip, and other terms known in the art. The sensor has biomolecules bound to a substrate surface as spots such that various complementary molecules can be bind to the biomolecules of the spots. In this description the molecules comprising the spots on the sensor will be referred to as probes. A probe is able to bind with a specific one or ones of target analytes whose presence in a sample is under inquiry. Probes are immobilized on a surface as circular spots, lines, patterns, or any other shape (the term “spot” as used in this description is intended to mean all forms of bioarrays for bioassay). The sensor is preferable constructed to have and be limited to probes that are complementary for binding with particular target analytes that are of interest. The construction of such sensors in general is well known in the art.

Another element is a labeled sample. The sample, as is usual in bioassay, is obtained from a source such as a blood, urine, saliva, serum, or any other source. The sample can also be from water, liquids from processes, or any other liquid in which an analyte target needs to be identified. The sample could also be solids, aerosols, or vapors that can be added to a liquid. The purpose is to determine if certain analytes are in the sample. The metal nanoparticle labels in the present invention in one specific embodiment are colloidal gold nanoparticles (CGNs). The process for labeling biomolecules to CGNs is described below as well as in the literature. In this invention at least two target analytes are being investigated and two sets of labels are used, one to attach to each target analyte, if it is present. The CGNs are selected in size groups to give visually distinguishable colors from each other as labels for each of the plurality of target analytes the presence of which is under inquiry. Also for best use the labels should be colorimetrically distinct.

A third element of the invention is an optical reader comprising a high power light source and possibly an optical scanner to detect and/or measure the colors and intensities of individual spots of the sensor after the sample has been exposed to the sensor and binding events have taken place so that the labeled target analytes are bound to their complementary probes.

In an embodiment of the invention CGNs to be used as labels are selected in at least two specific size groups. A size group is defined as a group that is calorimetrically distinct. Each size group must be calorimetrically distinguishable from the others used in the particular test. Different size groups are calorimetrically distinguishable from each other by eye or with an instrument. As will be appreciated, for good observation, the sizes selected should be as far apart colorimetrically as practical in order to result in the greatest color distinction. The CGNs are coated, or shelled preferably with a silica shell. The shell thickness should be sufficient that individual CGNs will remain so far apart that they will retain their calorimetric properties or will alter the colorimetric properties of its neighboring CGNs.

The shell thickness for each size group of CGNs may be the same so long as it is thick enough that it will be effective or can be different as long as it alters the properties of neighbors in a predictable fashion. A size group can be defined by the size of the CGNs, or by the shell thickness or both. CGNs are commercially available in discrete sizes to acceptable tolerances, so it is preferred that the group sizes be distinguished by different sized CGNs. In such case the shell thickness can be the same for all size groups since their calorimetric distinction would be caused by the different CGN sizes. Of course, the shell thicknesses could also be different for the different size groups. If the same size CGNs are to be used for all size groups then the differing colorimetric properties would have to established by different shell thicknesses for each size group.

A shelled CGN is a CGN that has a layer or coating of specific thickness surrounding the CGN such that the CGN exhibits size-dependent calorimetric properties. Any material can be used as the shell material so long as the dipole moment of the CGN is sufficiently altered that the CGNs will remain spaced apart to maintain their particular calorimetric properties or the CGN will alter the behavior of its neighboring particles but not lead to the properties of bulk gold. The principle is that if the field of influence of the CGN is sufficiently far from an adjacent CGN so that the fields do not influence each other, the original color will be maintained. Similarly, if the nanoparticles influence each other's field the colorimetric properties will be altered. In the extreme case, when the fields fully influence each other due to lack of a shell such as a silica shell keeping the nanoparticles apart, the CGNs behave as bulk gold and they lose their size-dependent properties. Preserving and tailoring of nanoparticles, in particular CGNs for exploitation of size-dependent calorimetric properties can be accomplished in a number of ways. For example different sized CGNs can be used for each group with large enough, but not necessarily uniform shells to isolate the field of influence. In this case precision of the shell thickness is less important because the CGNs will retain their colorimetric properties, each size group having its distinct and distinguishable colorimetric property. Alternatively, the CGNs could be the same size and the distinctive and distinguishable calorimetric property for each size group can be created by different sized shells. Other ways of using nanoparticle size and/or shell size to create a plurality of calorimetrically distinct and distinguishable groups will occur to those having skill in the art.

Before coating the CGNs with the preferred silica shell they are derivitized with a mercaptan-capping agent such as 3-mercaptopropionic acid, as described in Reference 1.

The silica is coated on derivitized CGNs based on the procedure described in the literature. In this approach, the CGNs are allowed to stand for varying periods of time in a sodium silicate solution. The particles are then centrifuged to remove free silicates. This method can be used to create shell thicknesses up to 4.6 nm. The Stober method can be used to create CGNs with thicker shells as also described in the literature. In this approach, silica coated CGNs will be concentrated, and a solution tetraethoxysilane is gradually added (drop wise) in an alkaline medium. This procedure is continued to create shelled CGNs (Si-CGN) of desired size.

The next step is to enable or functionalize the shelled CGNs for labeling by immobilizing on them reactive groups for the analyte under inquiry. Since each size group will be directed toward a different specific one or specific ones of the analytes, the reactive group must be reactive with the analyte or analytes for which that size group is designated. The methods used to derivitize the silica surface of the silica shelled CGNs with selected reactive groups are well known in the literature. The derivitized silica surfaces are immobilized with biomolecules such as antibodies, proteins, receptors, DNA or other materials. The biomolecules are selected such that they specifically bind to the one or more analytes whose presence in the sample is to be determined by the size group designated for that analyte or analytes. It should be appreciated that the generic definition of the size groups herein is to provide differently enabled nanoparticle groups that will have distinct and distinguishable colorimetric properties such that a plurality of analytes can be investigated simultaneously.

After the different groups have been enabled the sample containing the analytes is incubated with the enabled shelled CGNs and the analytes, if present, will bind to the reactive group biomolecules present on the surface of the silica shelled particles.

Now the sample is ready to be assayed by the sensor. The sample is exposed to the sensor. The analytes in the sample will bind to complimentary probes in the sensor. The biological material on the spots of the sensor are selected to bind with the specific one or ones of the analytes of interest. If the analyte or analytes are present, binding will occur. Since the analyte is attached to, that is labeled with, a shelled CGN, and adjacent shelled CGNs also bound to analytes at the probe site specific for that analyte are spaced sufficiently to preserve the colorimetric properties of the CGN, the binding event can be detected by color detection.

In an alternative procedure, the sample containing the analytes is applied over the sensor; binding will occur to complimentary immobilized probes. Then the enabled shelled CGNs are added to the sensor. The enabled shelled CGNs will attach to the specific analytes that have already bound to the probes for which they are active. This will form a sandwich and produce characteristic colors for the groups of shelled CGNs that have reactive groups for the analytes that are bound on the bioarray.

Reference colors can be provided to facilitate identification of the presence of analytes whose presence is under inquiry. The reference colors can be printed on the bioarray adjacent the spots that are conjugates for the analytes whose presence is under inquiry.

A key element of the invention is the use of a plurality of metal nanoparticle size groups, each size group being enabled for labeling a particular analyte of interest. This will allow for rapid repeated testing for particular biomolecules.

It should be understood that the foregoing disclosure includes certain specific embodiments of the invention and that all modifications and alternatives equivalent thereto are within the spirit and scope of the invention as set forth in the appended claims.

1. A method of multianalyte assay for a sample under investigation for the presence of a plurality of possible analytes comprising;
processing metal nanoparticles to add a shell to create a plurality of selected discrete size groups having distinguishable size dependent calorimetric properties;
enabling each shelled metal nanoparticle size group to be available for binding when mixed with the sample to a specific one or specific ones of the analytes whose presence is being tested for so as to label each analyte or selected analytes with a specific size group;
mixing the enabled shelled metal nanoparticle size groups with the sample to cause labeling of each of the specific one or specific ones of the possible analytes with the specific size group of shelled metal nanoparticles that has been enabled for labeling to that specific one or to the specific ones;
performing an assay of the sample of the type in which analytes bind to probe biomolecules for the analytes whose presence is being tested for;
calorimetrically observing the results of the assay to determine if any of the specific one or ones of the analytes being tested for is present.

2. The method of claim 1 in which the shelled nanoparticles are silica-shelled CGNs.

3. The method of claim 1 wherein the assay is performed by exposing the sample to a biochip array having immobilized probes suitable for binding with the plurality of analytes whose presence is being tested for.

4. The method of claim 1 wherein the step of calorimetrically observing comprises calorimetrically observing binding events in the biochip array for the presence of the analytes whose presence is being tested for.

5. The method of claim 2 in which the CGNs are of the same size and the size of the silica-shelled CGN groups is determined by variation of the silica shell.

6. The method of claim 2 in which the silica-shelled CGN groups are selected within a range of CGN sizes in which color differences are caused by quantum effects.

7. The method of claim 2 in which the silica-shelled CGN groups are selected within a range of CGN sizes in which color differences are caused by geometric effects.

8. The method of claim 2 in which the silica-shelled CGNs in a size group are separated by a spacing of more than twice the CGN radius.

9. The method of claim 2 in which the enabling step is that a reactive biomolecule specific for each target analyte of interest is immobilized on a selected size group of the silica-shelled CGNs to create an enabled CGN whereby when mixed with the sample, an analyte-CGN complex is formed and when the sample is brought into contact with the biochip array the complex forms a sandwich with the immobilized probes on the biochip array.

10. A colloidal metal nanoparticle bioarray, the bioarray formed on a substrate and having an array of probe sites said sites having selected different probe biomolecules and metal nanoparticles complexed with at least two of said different probe biomolecules said metal nanoparticle encased in a shell, the shell being of a thickness to impose a minimum separation between adjacent metal nanoparticles to allow calorimetrically distinct and distinguishable properties of the metal nanoparticles that are complexed with different probe biomolecules to be preserved.

11. The colloidal metal nanoparticle bioarray of claim 10 in which the metal nanoparticles are enabled to bind with selected probe biomolecules to establish calorimetrically distinct and distinguishable conjugates for different probes defined for different analytes.

12. The colloidal metal nanoparticle bioarray of claim 10 wherein the metal nanoparticles are shelled CGNs.

13. The colloidal metal nanoparticle bioarray of claim 12 wherein the CGNs are shelled with silica.

14. A method of multianalyte assay for a sample under investigation for the presence of a plurality of possible analytes comprising;
preparing silica-shelled CGNs in selected discrete size groups having distinguishable size dependent colorimetric properties and immobilizing on each size group a reactive biomolecule for one of the analytes whose presence is being investigated;
exposing the sample to a biochip array having immobilized probes for binding with the plurality of analytes whose presence is under investigation;
adding the silica-shelled CGNs having immobilized thereon the reactive biomolecules to form with specific target analytes a sandwich with spacing controlled by the silica-shelled CGNs to provide size dependent calorimetric distinction;
calorimetrically observing binding events in the biochip array for the presence of the analytes under investigation.

15. The method of claim 14 in which the CGNs are of the same size and the size of the silica-shelled CGN groups is determined by variation of the silica shell.

16. The method of claim 14 in which the silica-shelled CGN groups are selected within a range of CGN sizes in which color differences are caused by quantum effects.

17. The method of claim 14 in which the silica-shelled CGN groups are selected within a range of CGN sizes in which color differences are caused by geometric effects.

18. The method of claim 14 in which the silica-shelled CGNs in a size group are separated by a spacing of more than twice the CGN radius.

19. The method of claim 14 in which the enabling step is that a reactive biomolecule for each target analyte of interest is immobilized on a selected size group of the silica-shelled CGNs whereby when mixed with the sample a complex is formed and when the sample is brought into contact with the biochip array the complex forms a sandwich with the immobilized probes on the biochip array

20. A method of multianalyte assay for a sample under investigation for the presence of a plurality of possible different analyte types comprising;
combining the sample with prepared calorimetric labels in which each label is specific for one or ones of the analytes and each label being colorimetrically different to effect labeling of each analyte with the selected label for that analyte;
exposing the sample with labeled analytes, if any, to a sensor that has probe biomolecules that will bind to the analytes whose presence is under investigation;
observing the sensor for the presence of the calorimetrically labeled analytes.

21. A method of multianalyte assay for a sample under investigation for the presence of a plurality of possible analytes comprising;
starting with silica-shelled CGNs in a plurality of selected discrete size groups having distinguishable size dependent colorimetric properties;
enabling each size group of silica-shelled CGNs to be available for binding to label a specific one or specific ones of the analytes whose presence is being tested for;
labeling the enabled shelled CGNs with the specific one or specific ones of the possible analytes with the specific discrete size groups of shelled CGNs that have been enabled for binding to that specific one or to the specific ones;
exposing the labeled analytes to a sensor having probes for the analytes whose presence in the sample is under investigation;
examining the sensor by means of calorimetric detection for the calorimetrically different CGNs to detect whether any binding of analytes has occurred.

22. A method of multianalyte assay of a sample under investigation for the presence of a plurality of possible analytes comprising;
preparing metal nanoparticles as a label for each of the plurality of analytes whose presence is under investigation in a sample, the metal nanoparticles prepared as a label for each of said analytes having colorimetric properties that are distinguishable from the colorimetric properties of the metal nanoparticles prepared as a label for the others of said analytes;
labeling each of said plurality of analytes, if present, with the metal nanoparticles selected for labeling that analyte;
exposing the sample to a sensor having probes for the analytes whose presence is under investigation; and
examining the sensor calorimetrically for the metal nanoparticles to detect whether there has been binding to probes of any of the analytes whose presence is under investigation.
